(19) United States
(12) Patent Application Publication
(10) Pub. No.: US 2009/0253956 A1
(43) Pub. Date: Oct. 8, 2009

(54) CAPSULE ENDOSCOPE AND INTRA-STOMACH OBSERVING METHOD

(75) Inventor: Manabu FUJITA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/407,913

(22) Filed: Mar. 20, 2009

Related U.S. Application Data
(63) Continuation of application No. PCT/JP2007/068373, filed on Sep. 21, 2007.

(30) Foreign Application Priority Data
Sep. 22, 2006 (JP) 2006-257270

(51) Int. Cl. A61B 1/04

(52) U.S. Cl. 600/109

(57) ABSTRACT

A capsule endoscope includes a capsule casing which contains therein contents including an imaging unit and is inserted into an inside of a subject body; and a hollow volume changing unit which is connected to the capsule casing in an air-tight state, and changes a connection position with the capsule casing to change a volume of the capsule casing. The capsule endoscope also includes an actuator which is connected to the capsule casing and changes the connection position.

### CROSS-REFERENCE TO RELATED APPLICATIONS

This application is based upon and claims the benefit of priority from PCT Application Now PCT/JP2007/068373, filed on Sep. 21, 2007, the entire contents of which are incorporated herein by reference.

### BACKGROUND OF THE INVENTION

1. Field of the Invention

The present invention relates to a capsule endoscope which allows an observation of an inside of a subject body in a state of floating on a liquid fed into the subject body and an intra-stomach observing method.

2. Description of the Related Art

Recently, a capsule endoscope including an imaging function and a wireless communication function has appeared in the field of endoscopes. The capsule endoscope has a configuration of travelling inside of organs such as the esophagus, the stomach, and the small intestine (inside of a body cavity) according to their peristalsis and of sequentially capturing images by using the imaging function during an observation period which starts when the capsule endoscope is swallowed for the purpose of an observation (examination) from a mouth of a test subject as a subject body (human body) and ends when it is naturally excreted from a living body of the test subject.

Here, a technology of making a capsule endoscope, whose specific gravity is set to less than one and which is swallowed with a liquid (drinking water), float on the liquid inside the stomach to which the liquid is Led, and enabling an observation of a wall of the stomach is disclosed in International Publication Pamphlet No. 02/95351 (PCT National Publication No. 2004-529718).

However, the capsule endoscope houses contents including an imaging unit, an illumination unit, a wireless communication unit, a battery, and the like in a capsule casing; and a specific gravity of the contents is usually not less than one in most cases. Therefore, it is necessary to form the capsule casing in a size larger than the required size to make the specific gravity of the capsule endoscope less than one, so that the capsule endoscope has a problem of having a difficulty in swallowing from an oral cavity of the test subject as shown in International Publication Pamphlet No. 02/95351 (PCT National Publication No. 2004-529718).

### SUMMARY OF THE INVENTION

A capsule endoscope according to an aspect of the present invention includes a capsule casing which contains therein contents including an imaging unit and is inserted into an inside of a subject body; a hollow volume changing unit which is connected to the capsule casing, and changes a connection position with the capsule casing to change a volume of the capsule casing; and an actuator which is connected to the capsule casing and changes the connection position.

An intra-stomach observing method according to another aspect of the present invention includes making a capsule endoscope before an examination at a predetermined temperature; making a subject swallow the capsule endoscope; making the subject take in a water whose temperature is lower than the predetermined temperature; increasing a volume of the capsule endoscope; and observing an inside of a stomach by the capsule endoscope.

An intra-stomach observing method according to still another aspect of the present invention includes storing a capsule endoscope under an environment of a predetermined temperature; making a subject swallow the capsule endoscope; making the subject take in a water whose temperature is lower than the predetermined temperature; increasing a volume of the capsule endoscope; and observing an inside of a stomach by the capsule endoscope.

An intra-stomach observing method according to still another aspect of the present invention includes making a subject swallow a capsule endoscope; making the subject take in a water of a predetermined temperature and filling an inside of a stomach with a liquid; increasing a volume of the capsule endoscope; and observing the inside of the stomach by the capsule endoscope.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a view showing an observation of an inside of a subject body by using a capsule endoscope according to a first embodiment;

FIGS. 2A and 2B are schematic side views of examples of constitutions respectively before and after a volume increase of the capsule endoscope according to the first embodiment; and

FIGS. 3A and 3B are schematic side views of examples of constitutions respectively before and after a volume increase of a capsule endoscope according to a second embodiment.

### DETAILED DESCRIPTION OF THE PREFERRED EMBODIMENTS

Exemplary embodiments of a capsule endoscope will be explained below with reference to the accompanying drawings.

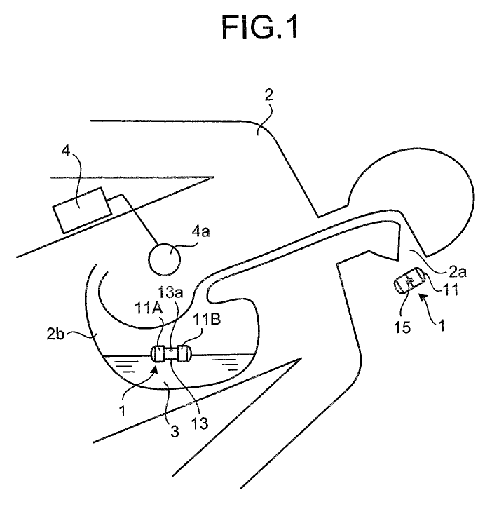

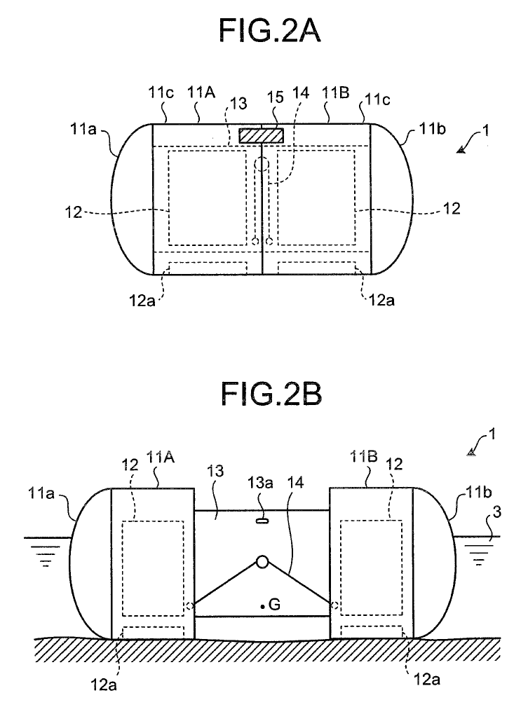

A first embodiment of the present invention will be explained first. FIG. 1 is a view showing an observation of an inside of a subject body by using a capsule endoscope according to a first embodiment and FIGS. 2A and 2B are schematic side views of examples of constitutions respectively before and after a volume increase of the capsule endoscope according to the first embodiment.

As shown in FIG. 1, a capsule endoscope 1 according to the first embodiment is inserted into a subject body 2 by being swallowed from an oral cavity 2a of the subject body 2, and images and observes a stomach wall as a target site by floating on a surface of water 3 whose specific gravity is one in a stomach 2b to which a liquid, for example, the water 3 is fed. A numeral 4 denotes a receiver which receives image data wirelessly transmitted from the capsule endoscope 1 having an imaging function and a wireless communication function. The receiver 4 includes a receiving antenna 4a such as a loop antenna which is attached on an outside surface of the subject body 2, and receives the image data and the like wirelessly transmitted from the capsule endoscope 1 via the receiving antenna 4a.

Here, the capsule endoscope 1 according to the first embodiment is explained by taking, as one example, an application to a compound-eye capsule endoscope which can capture images at both sides in a longitudinal axis direction of the capsule, and includes a capsule casing 11 whose size is small enough to be swallowed from the oral cavity 2a of the subject body 2 and contents 12 including an imaging unit, an illumination unit, a wireless communication unit, a substrate member, a battery, and the like which are embedded in the capsule casing 11.

The capsule casing 11 includes semispherical, transparent or translucent head covers 11a and 11b and a body cover 11c which has a cylindrical shape and is formed of a colored material which does not allow a visible light to pass through. The capsule casing 11 according to the embodiment, since the body cover 11c is divided into two pieces in the axial direction, is formed by separated two casings, that is, a first casing 11A and a second casing 11B Both of the divided surfaces of the divided body cover 11c are configured to be bottomed and to seal insides of the casings 11A and 11B in a liquid-tight manner, respectively.

The illumination unit in the contents 12 is constituted by a light emitting element such as an LED which emits an illumination light for illuminating a site to be imaged in the subject body 2 via the head covers 11a and 11b. The imaging unit in the contents 12 includes an imaging element such as a CCD and a CMOS sensor which receives a reflection light by the illumination light via the head covers 11a and 11b and captures images of the site to be imaged, an image forming lens, and the like. The contents 12 are separated and embedded into the two casings 11A and 11B respectively to fit a symmetrical structure of the compound-eye capsule endoscope, and each casing includes contents whose specific gravity in total is not less than one and higher than that of the water 3.

In addition to the configuration described above, the capsule endoscope 1 according to the embodiment includes a volume changing unit 13 and a shape memory alloy spring 14 as an actuator which activates the volume changing unit 13. The volume changing unit 13 is schematically a tank having a hollow rigid body of a cylindrical shape, arranged between the casings 11A and 11B with respect to the volume changing unit 13, and connected to be freely movable inward and outward with respect to the casings 11A and 11B along the longitudinal axis direction with an air-tight state with respect to the bottomed parts of the casings 11A and 11B maintained By this, the volume changing unit 13 can be displaced to a storage position where the volume changing unit 13 is stored half and half by the casings 11A and 11B as shown in FIG. 2A and to an exposure position where the volume changing unit 13 is exposed outward from the casings 11A and 11B as shown in FIG. 2B to increase a total casing volume of the capsule endoscope 1 and make the specific gravity less than one. To perform the displacement between the storage position and the exposure position, the volume changing unit 13 is provided, at a center part on a side surface, with one ventilation hole 13a through which an outer air is taken in and an inner air is discharged. Here, a heavy content in the contents 12, for example, a battery 12a is arranged on an inner wall opposite to the ventilation hole 13a in the casings 11A and 11B, so that a center of gravity G of the capsule endoscope 1 is set at a position where the ventilation hole 13a is oriented to an upper direction when the capsule endoscope 1 lies on its side.

The shape memory alloy spring 14, both ends thereof being connected to freely turn with respect to the bottomed parts of the casings 11A and 11B, causes the volume changing unit 13 to be displaced to the storage position by presenting a memorized shape of a folded shape as shown in FIG. 2A at a temperature around 36° C. to 40° C. corresponding to a body temperature of the subject body 2 and causes the volume changing unit 13 to be displaced to the exposure position by presenting a memorized shape of an expanded open shape as shown in FIG. 2B at a temperature around 25° C. corresponding to a temperature of the water 3 fed into the subject body 2. The shape memory alloy spring 14 is arranged to the side of the center of gravity G position away from the side of the ventilation hole 13a position.

Besides, the capsule endoscope 1 according to the embodiment includes, on a front surface, a sealing member 15 which seals the casings 11A and 11B to be a connected state so that the volume changing unit 13 is maintained at the storage position against the memorized shape of the shape memory alloy spring 14 that causes the displacement to the exposure position under a condition of a normal temperature before the capsule endoscope 1 is swallowed by the subject body 2. The sealing member 15 is formed of an edible material such as a wafer which dissolves by stomach juices and the like by being inserted into the subject body 2.

In this configuration, since the casings 11A and 11B are sealed by the sealing member 15, the volume changing unit 13 is maintained at the storage position against the memorized shape of the shape memory alloy spring 14 though the capsule endoscope 1 before starting an examination is under the condition of the normal temperature and the shape memory alloy spring 14 will try to present the memorized shape of the expanded open shape when the normal temperature is, for example, at about 25° C. By this, the capsule endoscope 1 can be maintained in a size of the state shown in FIG. 2A in which the casings 11A and 11B are coupled as one unit without causing the volume changing unit 13 to be exposed. The size is a normal capsule size and does not impair the swallowing performance from the oral cavity 2a.

At a time of starting the examination, the capsule endoscope 1 in the state as shown in FIG. 2A is swallowed from the oral cavity 2a and inserted into the stomach 2b. Upon the insertion into the subject body 2, the sealing member 15 dissolves by the stomach juices and the like and the sealed state is released. On this occasion, the capsule endoscope 1 is already inserted into the subject body 2 and subjected to a body temperature environment, the shape memory alloy spring 14 presents the memorized shape of the folded shape and therefore the volume changing unit 13 is maintained at the storage position even when the sealed state is released. By this, the capsule endoscope 1 can be maintained in the size of the state shown in FIG. 2A in which the casings 11A and 11B are coupled as one unit without causing the volume changing unit 13 to be exposed. In this state, the specific gravity of the capsule endoscope 1 is not less than one.

Thereafter, when the time is right, the water 3 at 25° C. is taken little by little to be fed into the stomach 2b. On this occasion, though the specific gravity of the capsule endoscope 1 is not less than one and the capsule endoscope 1 does not float on a surface of the water 3 fed into the stomach 2b, the capsule endoscope 1 becomes a state of lying on its side in which the ventilation hole 13a is oriented upward according to the position of the center of gravity G. Then, the shape memory alloy spring 14 of the lying capsule endoscope 1 comes to be present in the fed water 3 (the water 3 is assumed to be an amount not causing the capsule endoscope 1 to go under the water as shown in FIG. 2B at this point) and performs the displacement to present the memorized shape of the expanded open shape from the folded shape according to the temperature (25° C.) of the water 3. In response to the displacement to the expanded open shape of the shape memory alloy spring 14, the casings 11A and 11B are displaced to a direction of becoming away from each other, and the volume changing unit 13 connected between the casings 11A and 11B makes the displacement to the exposure position as shown in FIG. 2B while absorbing an air through a cylinder effect from the ventilation hole 13a present at a position oriented to an upper direction and in the air in the stomach 2b. Due to the displacement of the volume changing unit 13 to the exposure position, the casing volume of the capsule endoscope 1 increases to be a state where the specific gravity is less than one.

When an appropriate amount of the water 3 is further fed into the stomach 2b under this condition, the capsule endoscope 1 whose specific gravity is lowered to be less than one can image and observe the wall of the stomach while floating on the surface of the fed water 3 as shown in FIG. 1.

After the examination is completed, the water 3 is discharged from the inside of the stomach 2b to a side of the small intestine. By this, the capsule endoscope 1 remaining in the stomach 2b is subjected to the environment of the body temperature of the subject body 2 and the shape memory alloy spring 14 makes the displacement from the expanded open shape to a direction to be folded to present the memorized shape of the bent shape. In response to this folding displacement of the shape memory alloy spring 14, the separated casings 11A and 11B are also displaced to a direction to be in contact with each other and the volume changing unit 13 connected between the casings 11A and 11D also makes the displacement to the storage position as shown in FIG. 2A while discharging the inner air from the ventilation hole 13a. By the displacement of the volume changing unit 13 to the storage position, the size of the capsule endoscope 1 returns to a normal capsule size. Then, the capsule endoscope 1 moves to the side of the small intestine according to the subsequent peristalsis as usual and is finally excreted to an outside of the subject body 2.

As described, since the shape memory alloy spring 14 presents the memorized shape of causing the volume changing unit 13 to be displaced to the storage position where the volume changing unit 13 is stored in the capsule endoscope 1 based on the body temperature of the subject body 2 when the capsule endoscope 1 according to the embodiment is inserted into the subject body 2, a volume condition which does not impair the insertion property into the subject body 2 can be realized. Besides, since the shape memory alloy spring 14 presents the memorized shape of causing the volume changing unit 13 to be displaced to the exposure position where the volume changing unit 13 is exposed to the outside of the casing of the capsule endoscope 1 and the casing volume is increased based on the temperature of the water 3 fed appropriately into the subject body 2 after the capsule endoscope 1 is inserted into the subject body 2, the volume changing unit 13 makes the displacement to the exposure position to increase the casing volume while absorbing the outer air into the inside through the ventilation hole 13a in the subject body 2, so that an observation in a state where the capsule endoscope 1 with a low specific gravity in total floats on the water 3 can be performed even when the specific gravity of the contents 12 is high.

In addition, a common linear actuator may be provided instead of the shape memory alloy spring 14. A temperature sensor may further be equipped for controlling an operation of the linear actuator based on a temperature. By this, the volume changing unit 13 can be controlled with a greater flexibility.

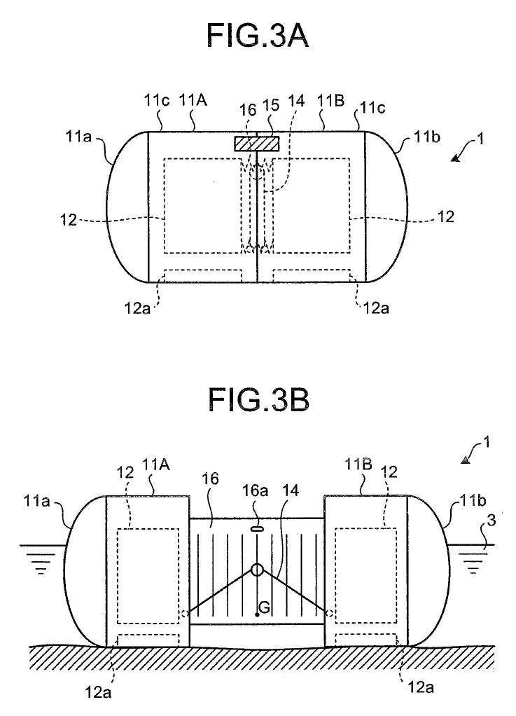

A second embodiment of the present invention will be explained next. FIGS. 3A and 3B are schematic side views of examples of constitutions respectively before and after a volume increase of a capsule endoscope according to a second embodiment. The same part as shown in the first embodiment is shown by using the same reference symbol.

In the second embodiment, a volume changing unit 16 which is a tank formed of a hollow soft member of an accordion shape is provided instead of the volume changing unit 13 which is a tank having a rigid body of a cylindrical shape. The volume changing unit 16 is arranged between the casings 11A and 11B with respect to the capsule casing 11 and connected, to be stretchable along the longitudinal axis direction, to the bottomed parts of the casings 11A and 11B with an air-tight state maintained. By this, the volume changing unit 16 can be displaced to a storage position where the volume changing unit 16 shrinks to be stored between the casings 11A and 11B as shown in FIG. 3A and to an exposure position where the volume changing unit 16 stretches out to be exposed outward from the casings 11A and 11B as shown in FIG. 3B to increase a total casing volume of the capsule endoscope 1 and make the specific gravity less than one. To perform the displacement between the storage position and the exposure position, the volume changing unit 16 is provided, at a center part on a side surface, with one ventilation hole 16a through which an outer air is taken in and an inner air is discharged. Other constituents are the same as those in the first embodiment.

In this configuration, since the casings 11A and 11B are sealed by the sealing member 15, the volume changing unit 16 is maintained at the storage position against the memorized shape of the shape memory alloy spring 14 though the capsule endoscope 1 before starting an examination is under the condition of the normal temperature and the shape memory alloy spring 14 will try to present the memorized shape of the expanded open shape when the normal temperature is, for example, at about 25° C. By this, the capsule endoscope 1 can be maintained in a size of the state shown in FIG. 3A in which the casings 11A and 11B are coupled as one unit without causing the volume changing unit 16 to be exposed. The size is a normal capsule size and does not impair the swallowing performance from the oral cavity 2a.

At a time of starting the examination, the capsule endoscope 1 in the state as shown in FIG. 3A is swallowed from the oral cavity 2a and inserted into the stomach 2b. Upon the insertion into the subject body 2, the sealing member 15 dissolves by the stomach juices and the like and the sealed state is released. On this occasion, the capsule endoscope 1 is already inserted into the subject body 2 and subjected to a body temperature environment, the shape memory alloy spring 14 presents the memorized shape of the folded shape and the volume changing unit 16 is maintained at the shrinking storage position even when the sealed state is released. By this, the capsule endoscope 1 can be maintained in the size of the state shown in FIG. 3A in which the casings 11A and 11B are coupled as one unit without causing the volume changing unit 16 to be exposed. In this state, the specific gravity of the capsule endoscope 1 is not less than one.

Thereafter, when the time is right, the water 3 at 25° C. is taken little by little to be fed into the stomach 2b. On this occasion, though the specific gravity of the capsule endoscope 1 is not less than one and the capsule endoscope 1 does not float on a surface of the water 3 fed into the stomach 2b, the capsule endoscope 1 becomes a state of lying on its side in which the ventilation hole 16a is oriented upward according to the position of the center of gravity G. Then, the shape memory alloy spring 14 of the lying capsule endoscope 1 comes to be present in the fed water 3 (the water 3 is assumed to be an amount not causing the capsule endoscope 1 to go under the water as shown in FIG. 3B at this point) and performs the displacement to present the memorized shape of the expanded open shape from the folded shape according to the temperature (25° C.) of the water 3. In response to the displacement to the expanded open shape of the shape memory alloy spring 14, the casings 11A and 11B are displaced to a direction of becoming away from each other and the volume changing unit 16 connected between the casings 11A and 11B also makes the displacement to the exposure position as shown in FIG. 3B while expanding by absorbing an outer air from the ventilation hole 16a present at a position oriented to an upper direction and in the air in the stomach 2b. Due to the displacement of the volume changing unit 16 to the exposure position according to the stretching, the casing volume of the capsule endoscope 1 increases to be a state where the specific gravity is less than one.

When an appropriate amount of the water 3 is further fed into the stomach 2b under this condition, the capsule endoscope 1 whose specific gravity is lowered to be less than one can image and observe the wall of the stomach while floating on the surface of the fed water 3 as shown in FIG. 1.

After the examination is completed, the water 3 is discharged from the inside of the stomach 2b to a side of the small intestine. By this, the capsule endoscope 1 remaining in the stomach 2b is subjected to the environment of the body temperature of the subject body 2 and the shape memory alloy spring 14 makes the displacement from the expanded open shape to a direction to be folded and present the memorized shape of the bent shape. In response to this folding displacement of the shape memory alloy spring 14, the separated casings 11A and 11B are also displaced to a direction to be in contact with each other and the volume changing unit 16 connected between the casings 11A and 11B also makes the displacement to the storage position as shown in FIG. 3A while discharging the inner air from the ventilation hole 16a. By the displacement of the volume changing unit 16 to the storage position according to the shrinking, the size of the capsule endoscope 1 returns to a normal capsule size Then, the capsule endoscope 1 moves to the side of the small intestine according to the subsequent peristalsis as usual and is finally excreted to an outside of the subject body 2. Hence, the case of the second embodiment shows the same advantageous effects as the case of the first embodiment.

Though the sealing by the sealing member 15 is adopted not to cause the capsule endoscope 1 before being swallowed to be displaced to a state of an increased volume in the first and the second embodiments, the capsule endoscope 1 may be, for example, stored under an environment of a constant temperature until a time right before being swallowed, so that the sealing member 15 may be eliminated Or more specifically, the subject body 2 may hold the capsule endoscope 1 by hand and the like right before the swallowing to keep the capsule endoscope 1 under the body temperature environment, so that the sealing member 15 may be eliminated.

Besides, when there is a possibility that the water 3 comes into the inside of the volume changing units 13 and 16 respectively through the ventilation holes 13a and 16a due to a rotation and the like of the capsule endoscope 1, a sheet member such as a Gore-Tex (registered trademark) which allows only a gas to path through may be attached onto the ventilation holes 13a and 16a respectively of the volume changing units 13 and 16 to prevent an intrusion of the water 3. In addition, the displacement operation to the exposure position may be configured to be performed through an adjustment based on an inner pressure of the capsule endoscope 1 without providing the ventilation holes 13a and 16a. By this, the structure can be simplified.

Moreover, though the explanation in the first and the second embodiments is made as an application example to the compound-eye capsule endoscope, a single-eye capsule endoscope is similarly applicable. Specifically, in the case of using the single-eye capsule endoscope, the capsule casing may not be divided into two pieces like the casings 11A and 11B, a volume changing unit may be connected, to be displaced at an air-tight state, to one end which is not the side of capturing images, and a shape memory alloy spring connecting a capsule casing and a distal end side of the volume changing unit may be provided.

A capsule endoscope and an intra-stomach observing method according to the present invention have advantageous effects that since an actuator changes a connecting position of a volume changing unit with a capsule casing based on a body temperature of a subject body when the capsule endoscope is inserted into the subject body to change a volume of the casing, a volume condition which does not impair a property of an insertion into the subject body can be realized, when a liquid is arbitrarily fed into the subject body after the capsule endoscope is inserted into the subject body, the actuator changes the connecting position of the volume changing unit with the capsule casing based on a temperature of the liquid to change and increase the casing volume, and thereby a total specific gravity can be reduced and an observation can be performed with the capsule endoscope floating on the liquid even when a specific gravity of contents is high.

Additional advantages and modifications will readily occur to those skilled in the art. Therefore, the invention in its broader aspects is not limited to the specific details and representative embodiments shown and described herein Accordingly, various modifications may be made without departing from the spirit or scope of the general inventive concept as defined by the appended claims and their equivalents.

What is claimed is:

1. A capsule endoscope, comprising:
a capsule casing which contains therein contents including an imaging unit and is inserted into an inside of a subject body;
a hollow volume changing unit which is connected to the capsule casing, and changes a connection position with the capsule casing to change a volume of the capsule casing; and
an actuator which is connected to the capsule casing and changes the connection position.

2. The capsule endoscope according to claim 1, wherein the actuator operates based on a change in a temperature of an outside of the capsule casing.

3. The capsule endoscope according to claim 1, wherein the actuator is a shape memory alloy spring.

4. The capsule endoscope according to claim 1, wherein
the connection position is a storage position where the volume of the capsule casing is minimum and an exposure position where the volume of the capsule casing is not minimum,
a specific gravity of the capsule casing with respect to a water is not less than one at the storage position, and
a specific gravity of the capsule casing with respect to the water is less than one at the exposure position.

5. The capsule endoscope according to claim 1, wherein the volume changing unit has a rigid body of a cylindrical shape and is connected to the capsule casing to be freely movable inward and outward with respect to the capsule casing.

6. The capsule endoscope according to claim 1, wherein the volume changing unit is formed of a soft member of an accordion shape and connected to be freely stretchable inward and outward with respect to the capsule casing.

7. The capsule endoscope according to claim 1, wherein
the capsule casing is constituted by divided two casings, and
the volume changing unit is connected between the two casings.

8. The capsule endoscope according to claim 7, wherein each of the two casings has a bottomed shape.

9. The capsule endoscope according to claim 4, wherein the actuator is formed by a shape memory alloy spring which causes the volume changing unit to be displaced from the storage position to the exposure position at a temperature not more than a predetermined temperature.

10. The capsule endoscope according to claim 9, further comprising a sealing member which is formed of a material that dissolves when inserted into the subject body and retains the volume changing unit at the storage position against a memorized shape, which causes the displacement to the exposure position, of the shape memory alloy spring.

11. The capsule endoscope according to claim 1, wherein a ventilation hole is formed in the volume changing unit.

12. The capsule endoscope according to claim 11, wherein a center of gravity of the capsule endoscope is set at a position where the ventilation hole is oriented upward with respect to a gravity direction.

13. The capsule endoscope according to claim 11, wherein a sheet member which allows only a gas to pass through is attached to the ventilation hole.

14. An intra-stomach observing method, comprising:
making a capsule endoscope before an examination at a predetermined temperature;
making a subject swallow the capsule endoscope;
making the subject take in a water whose temperature is lower than the predetermined temperature;
increasing a volume of the capsule endoscope; and
observing an inside of a stomach by the capsule endoscope.

15. The intra-stomach observing method according to claim 14, wherein the predetermined temperature is about 36° C. to 40° C. and the temperature lower than the predetermined temperature is about 25° C.

16. An intra-stomach observing method, comprising:
storing a capsule endoscope under an environment of a predetermined temperature;
making a subject swallow the capsule endoscope;
making the subject take in a water whose temperature is lower than the predetermined temperature;
increasing a volume of the capsule endoscope; and
observing an inside of a stomach by the capsule endoscope.

17. The intra-stomach observing method according to claim 16, wherein the predetermined temperature is about 36° C. to 40° C. and the temperature lower than the predetermined temperature is about 25° C.

18. An intra-stomach observing method, comprising:
making a subject swallow a capsule endoscope;
making the subject take in a water of a predetermined temperature and filling an inside of a stomach with a liquid;
increasing a volume of the capsule endoscope; and
observing the inside of the stomach by the capsule endoscope.
